(19) United States
(12) Patent Application Publication
(10) Pub. No.: US 2019/0055828 A1
(43) Pub. Date: Feb. 21, 2019

(54) SYSTEMS AND METHODS OF OPTIMIZING Y-GRADE NGL FRACTURING FLUIDS

(71) Applicants: John A. BABCOCK, (US); Linde Aktiengesellschaft, Munich (DE)

(72) Inventors: John A. BABCOCK, Houston, TX (US); Charles P. SIESS, III, Conroe, TX (US)

(21) Appl. No.: 15/680,749

(22) Filed: Aug. 18, 2017

(51) Int. Cl. E21B 43/267; E21B 43/25; E21B 47/00; G01N 33/22; G05B 17/00; C09K 8/94; C09K 8/90; C09K 8/88; C09K 8/80; C09K 8/70; C09K 8/64; C09K 8/60

(52) U.S. Cl. CPC E21B 43/267; E21B 43/255; E21B 47/00; G01N 33/225; G05B 17/00; C09K 8/602; C09K 8/90; C09K 8/887; C09K 8/80; C09K 8/703; C09K 8/64; C09K 8/94

(57) ABSTRACT

Fracturing fluids in the form of a hydrocarbon foam, an emulsion based foam, an emulsion, and a gelled fracturing fluid, each comprising Y-Grade NGL, which is an unfractionated hydrocarbon mixture that comprises ethane, propane, butane, isobutane, and pentane plus, wherein the unfractionated hydrocarbon mixture is a byproduct of a condensed and demethanized hydrocarbon stream.

### BACKGROUND

### Field of the Disclosure

Embodiments of this disclosure generally relate to systems and methods of optimizing Y-Grade NGL fracturing fluids.

### Description of the Related Art

Fracturing fluids are used to stimulate and improve fluid conductivity between a wellbore and a formation of interest to increase fluid production. There is a need, however, for fracturing fluids that are non-damaging to reservoir formations, have minimal water content and chemical additions, are naturally occurring and locally available, have fast clean-up, are cost effective, and are recoverable with minimal proppant flow back.

### SUMMARY

In one embodiment, a method of optimizing a Y-Grade NGL fracturing fluid comprises gathering geostatic data and reservoir fluid data of a hydrocarbon bearing reservoir; assessing availability of a supply of Y-Grade NGL and a gas; using the reservoir fluid data and data regarding the composition of the Y-Grade NGL and the gas to determine an equation of state; generating a hydrocarbon foam through a foam generation module, wherein the foam generation module includes customizing a surfactant to be mixed with the Y-Grade NGL and the gas to form the hydrocarbon foam, adjusting foam stability of the hydrocarbon foam, customizing the hydrocarbon foam, and determining a foam rheology of the hydrocarbon foam; formulating computational algorithms for the equation of state and the foam rheology; formulating a 3-D frac simulation model as represented by the geostatic data and the computational algorithms to simulate a hydraulic fracturing process of the reservoir using the hydrocarbon foam; and running multiple simulations for different hydrocarbon foams generated by the foam generation module to obtain an optimum propped frac length.

In one embodiment, a method of optimizing a Y-Grade NGL fracturing fluid comprises gathering geostatic data and reservoir fluid data of a hydrocarbon bearing reservoir; assessing availability of a supply of Y-Grade NGL, a gas, and water; using the reservoir fluid data and data regarding the composition of the Y-Grade NGL, the gas, and the water to determine an equation of state; generating an emulsion based foam through an emulsion based foam generation module, wherein the emulsion based foam generation module includes customizing a surfactant to be mixed with the Y-Grade NGL, the gas, and the water to form the emulsion based foam, adjusting foam stability of the emulsion based foam, customizing the emulsion based foam, and determining an emulsion based foam rheology of the emulsion based foam; formulating computational algorithms for the equation of state and the emulsion based foam rheology; formulating a 3-D frac simulation model as represented by the geostatic data and the computational algorithms to simulate a hydraulic fracturing process of the reservoir using the emulsion based foam; and running multiple simulations for different emulsion based foams generated by the emulsion based foam generation module to obtain an optimum propped frac length.

In one embodiment, a method of optimizing a Y-Grade NGL fracturing fluid comprises gathering geostatic data and reservoir fluid data of a hydrocarbon bearing reservoir; assessing availability of a supply of Y-Grade NGL and water; using the reservoir fluid data and data regarding the composition of the Y-Grade NGL and the water to determine an equation of state; generating an emulsion through an emulsion generation module, wherein the emulsion generation module includes customizing a surfactant to be mixed with the Y-Grade NGL and the water to form the emulsion, adjusting emulsion stability of the emulsion, customizing the emulsion, and determining an emulsion rheology of the emulsion; formulating computational algorithms for the equation of state and the emulsion rheology; formulating a 3-D frac simulation model as represented by the geostatic data and the computational algorithms to simulate a hydraulic fracturing process of the reservoir using the emulsion; and running multiple simulations for different emulsion generated by the emulsion generation module to obtain an optimum propped frac length.

In one embodiment, a method of optimizing a Y-Grade NGL fracturing fluid comprises gathering geostatic data and reservoir fluid data of a hydrocarbon bearing reservoir; assessing availability of a supply of Y-Grade NGL; using the reservoir fluid data and data regarding the composition of the Y-Grade NGL to determine an equation of state; generating a gelled fracturing fluid through a gel generation module, wherein the gel generation module includes customizing a gelling agent to be mixed with the Y-Grade NGL to form the gelled fracturing fluid, adjusting gel stability of the gelled fracturing fluid, customizing the gelled fracturing fluid, and determining a gel rheology of the gelled fracturing fluid; formulating computational algorithms for the equation of state and the gel rheology; formulating a 3-D frac simulation model as represented by the geostatic data and the computational algorithms to simulate a hydraulic fracturing process of the reservoir using the gelled fracturing fluid; and running multiple simulations for different gelled fracturing fluids generated by the gel generation module to obtain an optimum propped frac length.

### BRIEF DESCRIPTION OF THE DRAWINGS

So that the manner in which the above recited features can be understood in detail, a more particular description of the embodiments briefly summarized above may be had by reference to the embodiments below, some of which are illustrated in the appended drawings. It is to be noted, however, that the appended drawings illustrate only typical embodiments and are therefore not to be considered limiting of its scope, for the embodiments may admit to other equally effective embodiments.

FIG. 1 is a schematic view of a system for obtaining Y-Grade NGL according to one embodiment.

FIG. 2 is a schematic view of a method for optimizing hydrocarbon foam based Y-Grade NGL fracturing fluids according to one embodiment.

FIG. 3 is a schematic view of a method for optimizing emulsion based foam Y-Grade NGL fracturing fluids according to one embodiment.

FIG. 4 is a schematic view of a method for optimizing emulsion based Y-Grade NGL fracturing fluids according to one embodiment.

FIG. 5 is a schematic view of a method for optimizing gelled based Y-Grade NGL fracturing fluids according to one embodiment.

### DETAILED DESCRIPTION

Y-Grade natural gas liquids (referred to herein as Y-Grade NGL) is an un-fractionated hydrocarbon mixture comprising ethane, propane, butane, isobutane, and pentane plus. Pentane plus comprises pentane, isopentane, and/or heavier weight hydrocarbons, for example hydrocarbon compounds containing at least one of C5 through C8+. Pentane plus may include natural gasoline for example.

Typically, Y-Grade NGL is a by-product of condensed and de-methanized hydrocarbon streams that are produced from shale wells for example and transported to a centralized facility. Y-Grade NGL can be locally sourced from a splitter facility, a gas plant, and/or a refinery and transported by truck or pipeline to a point of use. In its un-fractionated or natural state (under certain pressures and temperatures, for example within a range of 250-600 psig and at wellhead or ambient temperature), Y-Grade NGL has no dedicated market or known use. Y-Grade NGL must undergo processing before its true value is proven.

The Y-Grade NGL composition can be customized for handling as a liquid under various conditions. Since the ethane content of Y-Grade NGL affects the vapor pressure, the ethane content can be adjusted as necessary. According to one example, Y-Grade NGL may be processed to have a low ethane content, such as an ethane content within a range of 3-12 percent by volume, to allow the Y-Grade NGL to be transported as a liquid in low pressure storage vessels. According to another example, Y-Grade NGL may be processed to have a high ethane content, such as an ethane content within a range of 38-60 percent by volume, to allow the Y-Grade NGL to be transported as a liquid in high pressure pipelines.

Y-Grade NGL differs from liquefied petroleum gas (“LPG”). One difference is that LPG is a fractionated product comprised of primarily propane, or a mixture of fractionated products comprised of propane and butane. Another difference is that LPG is a fractioned hydrocarbon mixture, whereas Y-Grade NGL is an unfractionated hydrocarbon mixture. Another difference is that LPG is produced in a fractionation facility via a fractionation train, whereas Y-Grade NGL can be obtained from a splitter facility, a gas plant, and/or a refinery. A further difference is that LPG is a pure product with the exact same composition, whereas Y-Grade NGL can have a variable composition.

In its unfractionated state, Y-Grade NGL is not an NGL purity product and is not a mixture formed by combining one or more NGL purity products. An NGL purity product is defined as an NGL stream having at least 90% of one type of carbon molecule. The five recognized NGL purity products are ethane (C2), propane (C3), normal butane (NC4 ), isobutane (IC4 ) and natural gasoline (C5+). The unfractionated hydrocarbon mixture is sent to a fractionation facility, where it is cryogenically cooled and passed through a fractionation train that consists of a series of distillation towers, referred to as deethanizers, depropanizers, and debutanizers, to fractionate out NGL purity products from the unfractionated hydrocarbon mixture. Each distillation tower generates an NGL purity product. Liquefied petroleum gas is an NGL purity product comprising only propane, or a mixture of two or more NGL purity products, such as propane and butane. Liquefied petroleum gas is therefore a fractionated hydrocarbon or a fractionated hydrocarbon mixture.

In one embodiment, Y-Grade NGL comprises 30-80%, such as 40-60%, for example 43%, ethane; 15-45%, such as 20-35%, for example 27%, propane; 5-10%, for example 7%, normal butane; 5-40%, such as 10-25%, for example 10%, isobutane; and 5-25%, such as 10-20%, for example 13%, pentane plus. Methane is typically less than 1%, such as less than 0.5% by liquid volume.

In one embodiment, Y-Grade NGL comprises condensed, dehydrated, desulfurized, and demethanized natural gas stream components that have a vapor pressure of not more than about 600 psig at 100 degrees Fahrenheit, with aromatics below about 1 weight percent, and olefins below about 1 percent by liquid volume. Materials and streams useful for the embodiments described herein typically include hydrocarbons with melting points below about 0 degrees Fahrenheit.

In one embodiment, Y-Grade NGL may be mixed with a chemical agent. The chemical agent may be mixed with a solubilizing fluid to liquefy any dry chemicals to aid in mixing with the Y-Grade NGL. The solubilizing fluid may comprise fractionated or refined hydrocarbons, such as C3, C4, C5, C6, C7, C8, C9, and mixtures thereof. The solubilizing fluid may comprise C3+ hydrocarbons, including propane, butane, pentane, naphtha, toluene, diesel, natural gasoline, and any combination thereof.

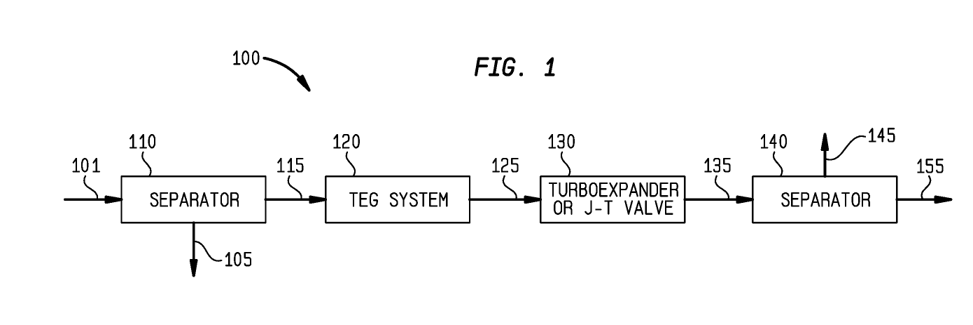

FIG. 1 is a schematic view of a Y-Grade NGL system 100 for obtaining Y-Grade NGL, according to one embodiment, for use with embodiments described herein. The system 100 includes a first separator 110, a triethylene glycol (“TEG”) system 120, a turboexpander 130 (or alternatively a Joule-Thompson valve), and a second separator 140. A hydrocarbon stream 101, such as a wet natural gas stream, flows into the first separator 110 where it is separated into a liquid stream 105 and a gas stream 115. The liquid stream 105 comprises liquid hydrocarbons and water. The gas stream 115 flows into the TEG system 120 where water vapor is removed to dehydrate the gas stream 115. The TEG system 120 dehydrates the gas stream 115 that is discharged from the first separator 110 to a water dew point up to −100 degrees Fahrenheit. The gas stream 125 exiting the TEG system 120 flows into the turboexpander 130 (or alternatively the Joule-Thompson valve), which cools the gas stream 125 to a temperature at or below 0 degrees Fahrenheit, for example to a temperature between 0 degrees Fahrenheit and −100 degrees Fahrenheit, for example about −30 degrees Fahrenheit.

The gas stream 125 is cooled to a temperature at or below 0 degrees Fahrenheit to condense out Y-Grade NGL from the remaining gas stream, which is primarily methane. The cooled fluids 135 flow into the second separator 140 where the gas stream 145, which is primarily methane, is separated out from the Y-Grade NGL 155. As a result, the Y-Grade NGL 155 is a byproduct of the condensed and de-methanized hydrocarbon stream 101.

In one embodiment, the gas stream 145 may also comprise ethane in an amount of about 1 percent to about 50 percent by volume. The amount of ethane separated out with the methane can be controlled by the pressure maintained in the second separator 140. The pressure in the second separator 140 may be about 600 psi or less. As the pressure is lowered in the second separator 140, the ethane content of the gas stream 145 is increased, and the ethane content of the Y-Grade NGL 155 is decreased. The Y-Grade NGL 155 may be used to form any of the fracturing fluids and/or with any of the systems and methods described herein.

According to one example, Y-Grade NGL comprises about 43% ethane, about 27% propane, about 7% normal butane, about 10% isobutane, and about 13% pentane plus at a maximum vapor pressure of about 600 psig at 100 degrees Fahrenheit per American Society for Testing and Materials (ASTM) according to the standard testing procedure D-6378 with methane, aromatics, and olefin maximums of 0.5% L.V. % per GPA 2177, 1.0 wt % of total stream per GPA 2186 and 1.0 L.V. % per GPA 2186, respectively.

According to one example, Y-Grade NGL comprises about 28% ethane, about 42% propane, about 13% normal butane, about 7% isobutane, and about 10% pentane plus. According to one example, Y-Grade NGL comprises about 48% ethane, about 31% propane, about 9% normal butane, about 5% isobutane, and about 7% pentane plus. According to one example, Y-Grade NGL comprises about 37%-43% ethane, about 22%-23% propane, about 7% normal butane, about 9%-11% isobutane, and about 13%-16% pentane plus. According to one example, Y-Grade NGL comprises about 10%-20% of at least one hydrocarbon compound having five carbon elements (C₅) or more.

Y-Grade NGL may comprise one or more combinations, as a whole or in part, of the Y-Grade NGL examples and/or embodiments described herein.

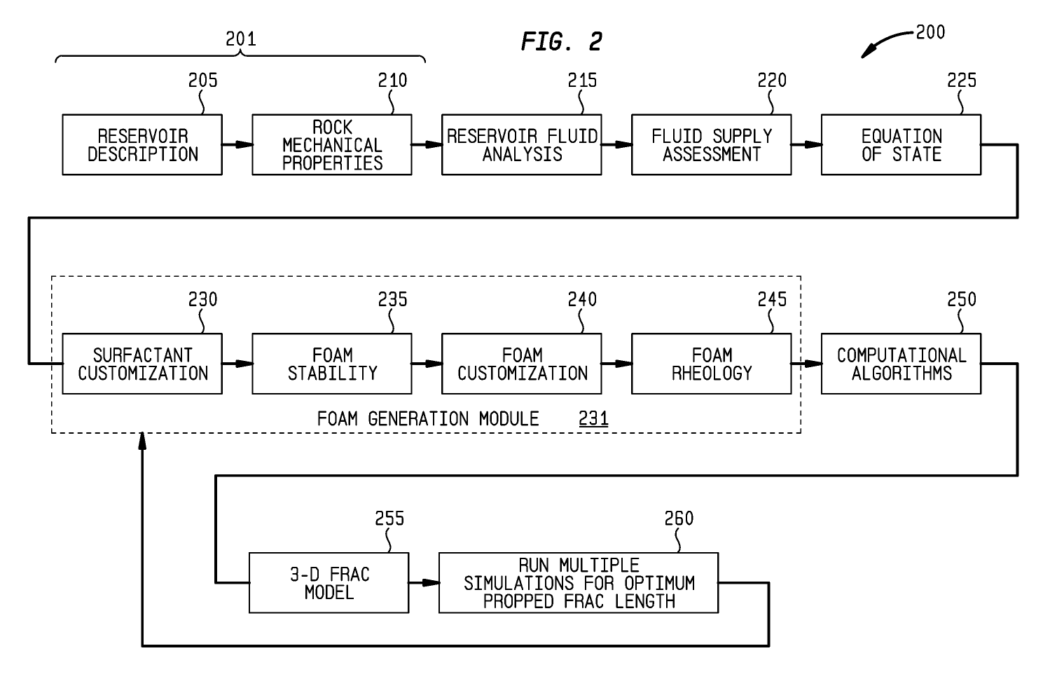

FIG. 2 is a schematic view of a method 200 for optimizing hydrocarbon foam based Y-Grade NGL fracturing fluids according to one embodiment.

The method 200 includes a step of gathering geostatic data 201 of a subsurface formation, such as a hydrocarbon bearing reservoir. Gathering the geostatic data 201 includes obtaining a description of the reservoir 205, including but not limited to data regarding porosity, permeability, fluid saturations, location, thickness, depth, pressures, and/or temperatures of the reservoir. Gathering the geostatic data 201 also includes conducting an analysis of the rock mechanical properties 210, including but not limited to data regarding Young's-modulus of elasticity and/or Poisson's ratio of the rock in the reservoir. The method 200 further includes a step of conducting a reservoir fluid analysis 215 to gather data regarding the reservoir fluid properties, including but not limited to composition, viscosity, density, gas/oil ratio, and/or water/oil ratio of the fluids in the reservoir.

The method 200 further includes a step of assessing the availability of a supply of fluids 220 needed to create the hydrocarbon foam based Y-Grade NGL fracturing fluids. To create hydrocarbon foam, the supply of fluids 220 will comprise Y-Grade NGL and a gas. The gas that can be used to form the hydrocarbon foam may comprise at least one of nitrogen, carbon dioxide, natural gas, methane, LNG, and ethane. An assessment is made to determine the quantity and/or composition of the Y-Grade NGL and/or the gas that is available for forming the hydrocarbon foam based Y-Grade NGL fracturing fluids.

The method 200 further includes a step of determining an equation of state 225 based on the reservoir fluid analysis 215 and the assessment of the supply of fluids 220. The equation of state 225 may be based on the reservoir fluid data and the quantity and/or composition of the Y-Grade NGL and/or the gas.

The method 200 further includes a step of generating a hydrocarbon foam through a foam generation module 231. The foam generation module 231 includes one or more of the steps of customizing a surfactant 230 to be mixed with the Y-Grade NGL and the gas to form the hydrocarbon foam, adjusting foam stability 235 of the hydrocarbon foam, customizing the hydrocarbon foam 240, and determining a foam rheology 245 of the hydrocarbon foam.

The step of customizing the surfactant 230 may include selecting a surfactant that is preferentially or selectively soluble in light hydrocarbons. Customizing the surfactant 230 may also include adjusting the molecular weight of the surfactant. Customizing the surfactant 230 may also include selecting at least one of a siloxane surfactant, a fluorosurfactant, a fatty acid ester, a glyceride, a silicon emulsifier, and a hydrophobic silica powder as the surfactant. The surfactant 230 may also be customized by adjusting the concentration of surfactant by up to 5% by weight of the liquid phase of the hydrocarbon foam.

The surfactant 230 may also be customized by selecting an anionic surfactant as the surfactant, wherein the anionic surfactant comprises at least one of 2-Acrylamido-2-methylpropane sulfonic acid, ammonium lauryl sulfate, ammonium perfluorononanoate, docusate, magnesium laureth sulfate, MBAS assay, perfluorobutanesulfonic acid, perfluorononanoic acid, perfluorooctanesulfonic acid, perfluorooctanoic acid, phospholipid, potassium lauryl sulfate, soap, soap substitute, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, and sulfolipid.

The step of adjusting foam stability 235 includes several ways of changing the stability of the foam. The stability of the foam may be adjusted by altering foam quality based on the amount of gas, such as nitrogen, that is used to form the hydrocarbon foam. The stability of the foam may be adjusted by adding nanoparticles to reduce fluid loss of the liquid phase of the hydrocarbon foam. The stability of the foam may be adjusted by adding a hydrocarbon soluble co-polymer to viscosify the liquid phase of the hydrocarbon foam. The stability of the foam may be adjusted by changing the type of gas used to form the hydrocarbon foam.

The step of adjusting foam stability 235 may also include adjusting the apparent viscosity of the foam. The apparent viscosity of the foam may be adjusted by altering foam quality based on the amount of gas, such as nitrogen, that is used to form the hydrocarbon foam. The apparent viscosity of the foam may be adjusted by adding a hydrocarbon soluble co-polymer to viscosify the liquid phase of the hydrocarbon foam. The apparent viscosity of the foam may be adjusted by adding a secondary fluid comprising up to 10% of the liquid phase of the hydrocarbon foam. The secondary fluid may comprise at least one of aromatics, alkanes, and crude oil.

The step of customizing the foam 240 includes adding a secondary fluid to the hydrocarbon foam, such as to control the mobility of the hydrocarbon foam in the reservoir. The secondary fluid may comprise at least one of aromatics, alkanes, and crude oil. Aromatics may comprise at least one of benzene, naphtha, xylene, toluene, fuel oils, olefins, and diesel. Alkanes may comprise at least one of heptane, octane, and hexane. Crude oil may comprise at least one of NGL's, condensate, light oil, and medium oil.

The step of determining foam rheology 245 includes determining the rheology of the hydrocarbon foam. The foam rheology 245 may be defined by, including but not limited to, its apparent viscosity, density, friction, flow rate, fluid loss, shear, and/or heat loss as a function of temperature, pressure, and/or composition of the hydrocarbon foam.

After the hydrocarbon foam is generated by the foam generation module 231 and the foam rheology 245 is determined, the method 200 further includes a step of formulating computational algorithms 250 for the equation of state 225 and the foam rheology 245. The method 200 further includes a step of formulating a 3-D frac simulation model 255 as represented by the geostatic data 201 and the computational algorithms 250 to simulate a hydraulic fracturing process of the reservoir using the hydrocarbon foam. The simulation will provide data regarding how the hydrocarbon foam is transported to the reservoir, how the hydrocarbon foam fractures the reservoir, and how the hydrocarbon foam transports proppant to the reservoir and places the proppant in the fractures in the reservoir.

After the simulation is run in the 3-D frac simulation model 255, the method 200 further includes a step running multiple simulations 260 by generating different types of hydrocarbon foams using the foam generation module 231 to gather enough data from the simulations to determine an optimum propped frac length and which hydrocarbon foam will achieve the optimum propped frac length in the reservoir. To achieve an optimum propped frac length in the reservoir, the hydrocarbon foam based Y-Grade NGL fracturing fluid is configured to create fractures in the reservoir having a desired height, width, and length, and distribute proppant within the fractures to maximize conductivity within the reservoir.

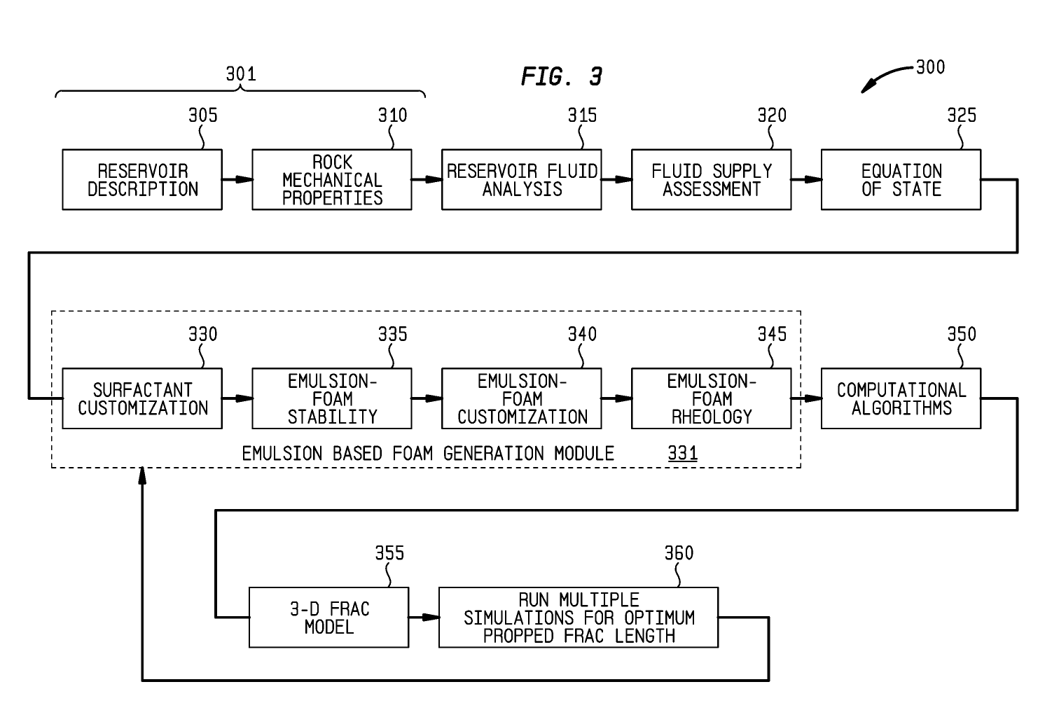

FIG. 3 is a schematic view of a method 300 for optimizing emulsion based foam Y-Grade NGL fracturing fluids according to one embodiment.

The method 300 includes the steps of gathering geostatic data 301 of a subsurface formation, such as a hydrocarbon bearing reservoir, and conducting a reservoir fluid analysis 315 to gather data regarding the reservoir fluid properties, similar to the steps of gathering geostatic data 201 and conducting the reservoir fluid analysis 215 in the method 200 described above.

The method 300 further includes a step of assessing the availability of a supply of fluids 320 needed to create the emulsion based foam Y-Grade NGL fracturing fluids. To create the emulsion based foam, the supply of fluids 320 will comprise Y-Grade NGL, a gas, such as nitrogen, and water. The gas that can be used to form the emulsion based foam may comprise at least one of nitrogen, carbon dioxide, natural gas, methane, LNG, and ethane. The water that can be used to form the emulsion based foam may be brine, seawater, and/or formation water, which comprises up to 10% of the liquid phase of the emulsion based foam. The water may also be fresh water inhibited with potassium chloride, which comprises up to 10% of the liquid phase of the emulsion based foam. The fresh water inhibited with potassium chloride may comprise up to 4% potassium chloride. An assessment is made to determine the quantity and/or composition of the Y-Grade NGL, the gas, and/or the water that is available for forming the emulsion based foam Y-Grade NGL fracturing fluids.

The method 300 further includes a step of determining an equation of state 325 based on the reservoir fluid analysis 315 and the assessment of the supply of fluids 320. The equation of state 325 may be based on the reservoir fluid data and the quantity and/or composition of the Y-Grade NGL, the gas, and/or the water.

The method 300 further includes a step of generating an emulsion based foam through an emulsion based foam generation module 331. The emulsion based foam generation module 331 includes one or more of the steps of customizing a surfactant 330 to be mixed with the Y-Grade NGL, the gas, and the water to form the emulsion based foam, adjusting emulsion based foam stability 335 of the emulsion based foam, customizing the emulsion based foam 340, and determining a emulsion based foam rheology 345 of the emulsion based foam.

The step of customizing the surfactant 330 may include selecting a surfactant that is preferentially or selectively soluble in light hydrocarbons. For emulsion based foams, the surfactant 330 acts as a foaming agent, an emulsifying agent, or both. Customizing the surfactant 330 may also include adjusting the molecular weight of the surfactant. The surfactant 330 may also be customized by adjusting the concentration of surfactant by up to 5% by weight of the liquid phase of the emulsion based foam. Customizing the surfactant 330 may also include selecting at least one of a non-ionic surfactant, an anionic surfactant, and a cationic surfactant as the surfactant.

The non-ionic surfactant comprises at least one of a siloxane, a fluorosurfactant, a fatty acid ester, a glyceride, and a silicon emulsifier, and a hydrophobic silica powder.

The anionic surfactant comprises at least one of 2-Acrylamido-2-methylpropane sulfonic acid, ammonium lauryl sulfate, ammonium perfluorononanoate, docusate, magnesium laureth sulfate, MBAS assay, perfluorobutanesulfonic acid, perfluorononanoic acid, perfluorooctanesulfonic acid, perfluorooctanoic acid, phospholipid, potassium lauryl sulfate, soap, soap substitute, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, and sulfolipid.

The cationic surfactant comprises at least one of behentrimonium chloride, benzalkonium chloride, benzethonium chloride, bronidox, cetrimonium bromide, cetrimonium chloride, dimethyldioctadecylammonium bromide, dimethyldioctadecylammonium chloride, lauryl methyl gluceth-10 hydroxypropyl dimonium chloride, octenidine dihydrochloride, olaflur, N-Oleyl-1,3-propanediamine, stearalkonium chloride, tetramethylammonium hydroxide, and thonzonium bromide.

The step of adjusting emulsion based foam stability 335 includes several ways of changing the stability of the emulsion based foam. The stability of the emulsion based foam may be adjusted by altering foam quality based on the amount of gas, such as nitrogen, that is used to form the emulsion based foam. The stability of the emulsion based foam may be adjusted by adding nanoparticles to reduce fluid loss of the liquid phase of the emulsion based foam. The stability of the emulsion based foam may be adjusted by adding a hydrocarbon soluble co-polymer to viscosify the liquid phase of the emulsion based foam. The stability of the emulsion based foam may be adjusted by changing the type of gas used to form the emulsion based foam. The emulsion based foam stability may be adjusted by adding a water soluble co-polymer to viscosify the liquid phase of the emulsion based foam.

The step of adjusting emulsion based foam stability 335 may also include adjusting the apparent viscosity of the emulsion based foam. The apparent viscosity of the emulsion based foam may be adjusted by changing foam quality based on the amount of gas, such as nitrogen, that is used to form the emulsion based foam. The apparent viscosity of the emulsion based foam may be adjusted by adding a hydrocarbon soluble co-polymer to viscosify the liquid phase of the emulsion based foam. The apparent viscosity of the emulsion based foam may be adjusted by adding a secondary fluid comprising up to 10% of the liquid phase of the emulsion based foam. The secondary fluid may comprise at least one of aromatics, alkanes, and crude oil. The apparent viscosity of the emulsion based foam may be adjusted by adding a water soluble co-polymer to viscosify the liquid phase of the emulsion based foam.

The step of customizing the emulsion based foam 340 includes adding a secondary fluid to the emulsion based foam. The secondary fluid may comprise at least one of aromatics, alkanes, and crude oil. Aromatics may comprise at least one of benzene, naphtha, xylene, toluene, fuel oils, olefins, and diesel. Alkanes may comprise at least one of heptane, octane, and hexane. Crude oil may comprise at least one of NGL's, condensate, light oil, and medium oil.

The step of determining emulsion based foam rheology 345 includes determining the rheology of the emulsion based foam. The emulsion based foam rheology 345 may be defined by, including but not limited to, its apparent viscosity, density, friction, flow rate, fluid loss, shear, and/or heat loss as a function of temperature, pressure, and/or composition of the emulsion based foam.

After the emulsion based foam is generated by the emulsion based foam generation module 331 and the emulsion based foam rheology 345 is determined, the method 300 further includes a step of formulating computational algorithms 350 for the equation of state 325 and the emulsion based foam rheology 345. The method 300 further includes a step of formulating a 3-D frac simulation model 355 as represented by the geostatic data 301 and the computational algorithms 350 to simulate a hydraulic fracturing process of the reservoir using the emulsion based foam. The simulation will provide data regarding how the emulsion based foam is transported to the reservoir, how the emulsion based foam fractures the reservoir, and how the emulsion based foam transports proppant to the reservoir and places the proppant in the fractures in the reservoir.

After the simulation is run in the 3-D frac simulation model 355, the method 300 further includes a step running multiple simulations 360 of different types of emulsion based foams generated by the emulsion based foam generation module 331 to gather enough data from the simulations to determine an optimum propped frac length and which emulsion based foam will achieve the optimum propped frac length in the reservoir. To achieve an optimum propped frac length in the reservoir, the emulsion based foam Y-Grade NGL fracturing fluid is configured to create fractures in the reservoir having a desired height, width, and length, and distribute proppant within the fractures to maximize conductivity within the reservoir.

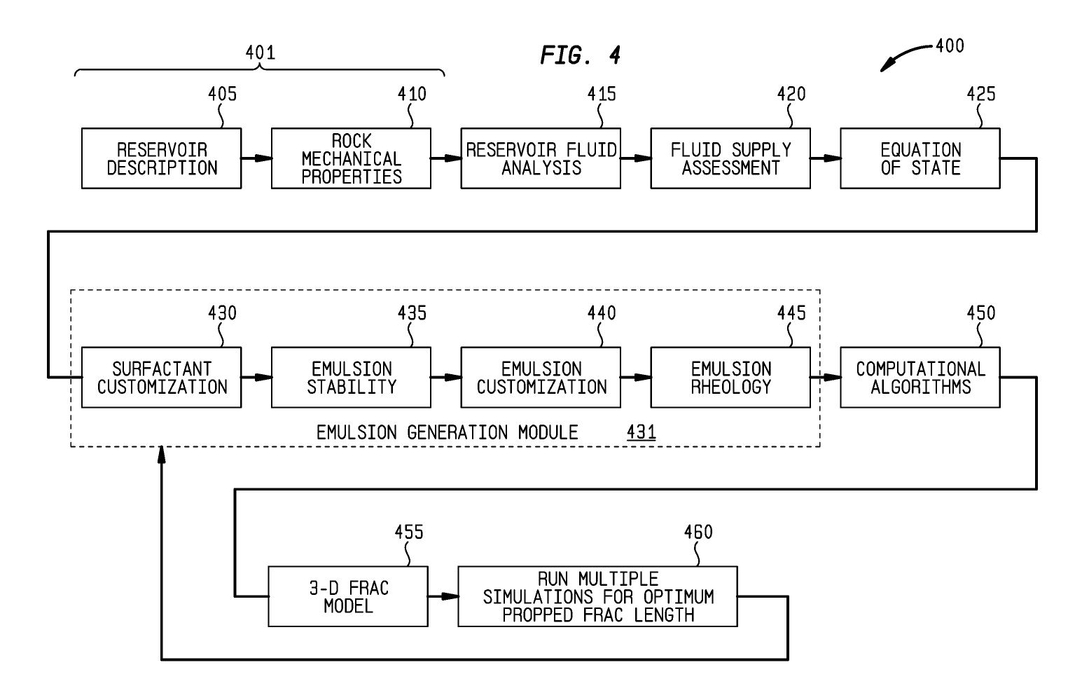

FIG. 4 is a schematic view of a method 400 for optimizing emulsion based Y-Grade NGL fracturing fluids according to one embodiment.

The method 400 includes the steps of gathering geostatic data 401 of a subsurface formation, such as a hydrocarbon bearing reservoir, and conducting a reservoir fluid analysis 415 to gather data regarding the reservoir fluid properties, similar to the steps of gathering geostatic data 201 and conducting the reservoir fluid analysis 215 in the method 200 described above.

The method 400 further includes a step of assessing the availability of a supply of fluids 420 needed to create the emulsion based Y-Grade NGL fracturing fluids. To create an emulsion, the fluids 420 will comprise Y-Grade NGL and water. The water that can be used to form the emulsion may be brine, seawater, and/or formation water, which comprises up to 10% of the liquid phase of the emulsion. The water may also be fresh water inhibited with potassium chloride, which comprises up to 10% of the liquid phase of the emulsion. The fresh water inhibited with potassium chloride may comprise up to 4% potassium chloride. An assessment is made to determine the quantity and/or composition of the Y-Grade NGL and/or the water that is available for forming the emulsion based Y-Grade NGL fracturing fluids.

The method 400 further includes a step of determining an equation of state 425 based on the reservoir fluid analysis 415 and the assessment of the supply of fluids 420. The equation of state 425 may be based on the reservoir fluid data and the quantity and/or composition of the Y-Grade NGL and/or the water.

The method 400 further includes a step of generating an emulsion through an emulsion generation module 431. The emulsion generation module 431 includes one or more of the steps of customizing a surfactant 430 to be mixed with the Y-Grade NGL and the water to form the emulsion, adjusting emulsion stability 435 of the emulsion, customizing the emulsion 440, and determining a emulsion rheology 445 of the emulsion.

The step of customizing the surfactant 430 may include selecting a surfactant that is preferentially or selectively soluble in light hydrocarbons. For emulsions, the surfactant 430 acts as an emulsifying agent. Customizing the surfactant 430 may also include adjusting the molecular weight of the surfactant. The surfactant 430 may also be customized by adjusting the concentration of surfactant by up to 5% by weight of the emulsion. Customizing the surfactant 430 may also include selecting at least one of a non-ionic surfactant, an anionic surfactant, and a cationic surfactant as the surfactant.

The non-ionic surfactant comprises at least one of a siloxane, a fluorosurfactant, a fatty acid ester, a glyceride, a silicon emulsifier, and a hydrophobic silica powder.

The anionic surfactant comprises at least one of 2-Acrylamido-2-methylpropane sulfonic acid, ammonium lauryl sulfate, ammonium perfluorononanoate, docusate, magnesium laureth sulfate, MBAS assay, perfluorobutanesulfonic acid, perfluorononanoic acid, perfluorooctanesulfonic acid, perfluorooctanoic acid, phospholipid, potassium lauryl sulfate, soap, soap substitute, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, and sulfolipid.

The cationic surfactant comprises at least one of behentrimonium chloride, benzalkonium chloride, benzethonium chloride, bronidox, cetrimonium bromide, cetrimonium chloride, dimethyldioctadecylammonium bromide, dimethyldioctadecylammonium chloride, lauryl methyl gluceth-10 hydroxypropyl dimonium chloride, octenidine dihydrochloride, olaflur, N-Oleyl-1,3-propanediamine, stearalkonium chloride, tetramethylammonium hydroxide, and thonzonium bromide.

The step of adjusting emulsion stability 435 includes several ways of changing the stability of the emulsion. The stability of the emulsion may be adjusted by changing the percent volume of water used to form the emulsion. The stability of the emulsion may be adjusted by adding a viscosifier to the emulsion. The viscosifier may comprise at least one of a hydrocarbon soluble co-polymer and a water soluble viscosifier. The water soluble viscosifer may comprise at least one of water soluble co-polymers, polysaccarides, guar gum, viscoelastic surfactants, crosslinkers, cellulosic viscosifiers, and hydroxyethyl cellulos.

The step of adjusting emulsion stability 435 may also include adjusting the apparent viscosity of the emulsion. The apparent viscosity of the emulsion may be adjusted by adding a hydrocarbon soluble or water soluble co-polymer to viscosify the liquid phase of the emulsion. The apparent viscosity of the emulsion may be adjusted by changing the percent volume of water used to form the emulsion. The apparent viscosity of the emulsion may be adjusted by adding a secondary fluid comprising up to 10% of the liquid phase of the emulsion. The secondary fluid may comprise at least one of aromatics, alkanes, and crude oil.

The step of customizing the emulsion 440 includes adding a secondary fluid to the emulsion. The secondary fluid may comprise at least one of aromatics, alkanes, and crude oil. Aromatics may comprise at least one of benzene, naphtha, xylene, toluene, fuel oils, olefins, and diesel. Alkanes may comprise at least one of heptane, octane, and hexane. Crude oil may comprise at least one of NGL's, condensate, light oil, and medium oil.

The step of determining emulsion rheology 445 includes determining the rheology of the emulsion. The emulsion rheology 445 may be defined by, including but not limited to, its apparent viscosity, density, friction, flow rate, fluid loss, shear, and/or heat loss as a function of temperature, pressure, and/or composition of the emulsion.

After the emulsion is generated by the emulsion generation module 431 and the emulsion rheology 445 is determined, the method 400 further includes a step of formulating computational algorithms 450 for the equation of state 425 and the emulsion rheology 445. The method 400 further includes a step of formulating a 3-D frac simulation model 455 as represented by the geostatic data 401 and the computational algorithms 450 to simulate a hydraulic fracturing process of the reservoir using the emulsion. The simulation will provide data regarding how the emulsion is transported to the reservoir, how the emulsion fractures the reservoir, and how the emulsion transports proppant to the reservoir and places the proppant in the fractures in the reservoir.

After the simulation is run in the 3-D frac simulation model 455, the method 400 further includes a step running multiple simulations 460 of different types of emulsions generated by the emulsion generation module 431 to gather enough data from the simulations to determine an optimum propped frac length and which emulsion will achieve the optimum propped frac length in the reservoir. To achieve an optimum propped frac length in the reservoir, the emulsion based Y-Grade NGL fracturing fluid is configured to create fractures in the reservoir having a desired height, width, and length, and distribute proppant within the fractures to maximize conductivity within the reservoir.

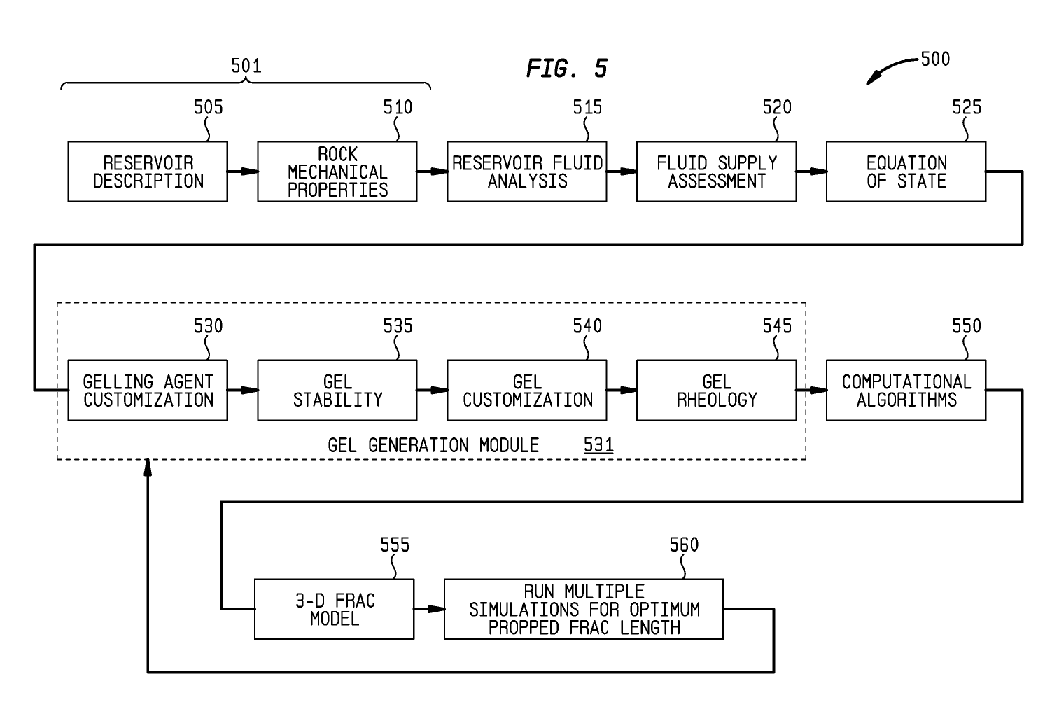

FIG. 5 is a schematic view of a method 500 for optimizing gelled based Y-Grade NGL fracturing fluids according to one embodiment.

The method 500 includes the steps of gathering geostatic data 501 of a subsurface formation, such as a hydrocarbon bearing reservoir, and conducting a reservoir fluid analysis 515 to gather data regarding the reservoir fluid properties, similar to the steps of gathering geostatic data 201 and conducting a reservoir fluid analysis 215 in the method 200.

The method 500 further includes a step of assessing the availability of a supply of fluids 520 needed to create the gelled based Y-Grade NGL fracturing fluids. To create a gelled fracturing fluid, the fluids 520 will comprise Y-Grade NGL and a gelling agent. An assessment is made to determine the quantity and/or composition of the Y-Grade NGL and/or the gelling agent that is available for forming the gelled based Y-Grade NGL fracturing fluids.

The method 500 further includes a step of determining an equation of state 525 based on the reservoir fluid analysis 515 and the assessment of the supply of fluids 520. The equation of state 525 may be based on the reservoir fluid data and the quantity and/or composition of the Y-Grade NGL and/or the gelling agent.

The method 500 further includes a step of generating a gelled fracturing fluid through a gel generation module 531. The gel generation module 531 includes one or more of the steps of customizing a gelling agent 530 to be mixed with the Y-Grade NGL to form the gelled fracturing fluid, adjusting gel stability 535 of the gelled fracturing fluid, customizing the gelled fracturing fluid 540, and determining a gel rheology 545 of the gelled fracturing fluid.

The step of customizing the gelling agent 530 may include selecting at least one of hydrocarbon soluble copolymers, phosphate esters, organo-metallic complex cross-linkers, amine carbamates, aluminum soaps, cocoamine (C12-C14), sebacoyl chloride, oley (C18) amine, toulen-2, 4-diisocyanate, tolune-2, 6-diisolcyanate, and combinations thereof as the gelling agent.

The step of adjusting gel stability 535 includes several ways of changing the stability of the gelled fracturing fluid. The stability of the gelled fracturing fluid may be adjusted by changing at least one of the type of gelling agent and the concentration of the gelling agent used to form the gelled fracturing fluid.

The step of customizing the gelled fracturing fluid 540 includes adding a secondary fluid to the gelled fracturing fluid. The secondary fluid may comprise at least one of aromatics, alkanes, and crude oil. Aromatics may comprise at least one of benzene, naphtha, xylene, toluene, fuel oils, olefins, and diesel. Alkanes may comprise at least one of heptane, octane, and hexane. Crude oil may comprise at least one of NGL's, condensate, light oil, and medium oil.

The step of determining gel rheology 545 includes determining the rheology of the gelled fracturing fluid. The gel rheology 545 may be defined by, including but not limited to, its apparent viscosity, density, friction, flow rate, fluid loss, shear, and/or heat loss as a function of temperature, pressure, and/or composition of the gelled fracturing fluid.

After the gelled fracturing fluid is generated by the gel generation module 531 and the gel rheology 545 is determined, the method 500 further includes a step of formulating computational algorithms 550 for the equation of state 525 and the gel rheology 545. The method 500 further includes a step of formulating a 3-D frac simulation model 555 as represented by the geostatic data 501 and the computational algorithms 550 to simulate a hydraulic fracturing process of the reservoir using the gelled fracturing fluid. The simulation will provide data regarding how the gelled fracturing fluid is transported to the reservoir, how the gelled fracturing fluid fractures the reservoir, and how the gelled fracturing fluid transports proppant to the reservoir and places the proppant in the fractures in the reservoir.

After the simulation is run in the 3-D frac simulation model 555, the method 500 further includes a step running multiple simulations 560 of different types of gelled fracturing fluids generated by the gel generation module 531 to gather enough data from the simulations to determine an optimum propped frac length and which gelled fracturing fluid will achieve an optimum propped frac length in the reservoir. To achieve an optimum propped frac length in the reservoir, the gelled based Y-Grade NGL fracturing fluid is configured to create fractures in the reservoir having a desired height, width, and length, and distribute proppant within the fractures to maximize conductivity within the reservoir.

The supply of Y-Grade NGL may be provided in Y-Grade NGL storage tanks that comprise of onsite Y-Grade NGL pressurized storage vessels that are supplied from a regional Y-Grade NGL gathering pipeline, a regional gas splitter, or a gas processing facility via tanker trucks. The supply of proppant can be temporally stored in the pressurized proppant silo and pneumatically conveyed using a pressurized gas, such as nitrogen and/or carbon dioxide.

The fracturing fluids disclosed herein may be injected into a subsurface formation, such as a hydrocarbon bearing reservoir, at a pressure that overcomes the rock mechanical properties of the rock of the subsurface formation to fracture the rock formation. In some cases, the pressure needed to fracture the rock formation is about 7,000 psig, but pressures may exceed 10,000 psig to create fractures at greater depths. As the fracturing fluid is pumped into the rock formation, pressure builds as the rock formation is pressurized with the fracturing fluid and flow areas become increasingly restricted until the natural stress within the rock formation is exceeded. When pressure within the rock formation reaches a critical point, sometimes referred to as “breakdown pressure,” fractures begin to nucleate and grow within the rock formation. When the rock formation begins to yield, pressure may drop to a fracture propagation range.

The fracturing fluids disclosed herein may be injected into a subsurface formation at a temperature that cools and lowers the temperature of the rock of the subsurface formation to fracture the rock formation. In some cases, the rock of the subsurface formation may have been previously hydraulically fractured and the cooling (or thermal shock) provided by the fracturing fluid creates additional fractures in the rock of the subsurface formation. In some cases, the fracturing fluid may be injected into the subsurface formation at a temperature that cools and lowers the temperature of the rock of the subsurface formation but does not fracture the rock formation.

The fracturing fluids disclosed herein may comprise a proppant. The proppant supplied from the proppant storage unit, the pressurized proppant blender unit, or the pressurized proppant system may be optionally added to any of the fracturing fluids. The proppant may include sand and/or ceramic materials. The proppant supplied may be “bone dry” (e.g. substantially free from any liquid, such as water) when mixed to form the fracturing fluid.

The fracturing fluids, such as the hydrocarbon foam, the emulsion based foam, the emulsion, and the gelled fracturing fluids, disclosed herein may comprise non-aqueous based chemical agents. The non-aqueous based chemical agents include but are not limited to non-aqueous based foaming agents, foam stabilizers, emulsifying agents, gelling agents, viscosity increasing agents, surfactants, nanoparticles, and combinations thereof.

The fracturing fluids, such as the emulsion based foam and the emulsion, disclosed herein may comprise aqueous based chemical agents. The aqueous based chemical agents include but are not limited to aqueous based foaming agents, foam stabilizers, emulsifying agents, gelling agents, viscosity increasing agents, surfactants, nanoparticles, breakers, friction reducers, scale inhibiters, biosides, acids, buffer/pH adjusting agents, clay stabilizers, corrosion inhibiters, crosslinkers, iron controls, solvents, and combinations thereof.

The fracturing fluids, such as the hydrocarbon foam and the emulsion based foam, disclosed herein may comprise foaming agents. The foaming agents include but are not limited to nonionic surfactants, wherein the nonionic surfactants comprise at least one of a siloxane surfactant, a fluorosurfactant, a fatty acid ester, a glyceride, a silicon emulsifier, a hydrophobic silica powder, and combinations thereof.

The foaming agents for the emulsion based foam may also include but are not limited to surfactants, such as ionic surfactants, nonionic surfactants, anionic surfactants, cationic surfactants, iC90-glycol, iC10-glycol, 1-propanol, iso-propanol, 2-butanol, butyl glycol, sulfonic acids, betaine compounds, fluorosurfactants, hydrocarbon solvents, aluminum soaps, phosphate esters, alcoholethersulfates, alcohol sulfate, alcylsulfates, isethionates, sarconisates, acylsarcosinates, olefinsulfonates, alcylethercarboxylates, alcylalcoholamides, aminoxids, alkylbenzolsulfonate, alkylnaphthalene sulfonates, fattyalcohol ethoxylates, oxo-alcohol ethoxylates, alkylethoxylates, alkylphenolethoxylates, fattyamin- and fattyamidethoxylates, alkylpolyglucosides, oxoalcohol ethoxylates, guerbetalcohol alkoxylates, alkylethersulfonate, EO/PO blockpolymers, betaines, cocamidopropylbetaine, C8-C10 alkylamidopropylbetaine, sulfobetaines, alkenylsulfonates, alkylglykols, alcoholalkoxylates, sulfosuccinates, alkyletherphosphates, esterquats, dialcylammoniumderivatives, trialcylammoniumderivatives, and combinations thereof.

The fracturing fluids, such as the hydrocarbon foam and the emulsion based foam, disclosed herein may comprise foam stabilizers. The foam stabilizers include but are not limited to proteins, microparticles, nanoparticles, silica, and silica derivatives that are known to stabilize foam and emulsions through so-called “pickering”. The foam stabilizers may comprise additives that increase the viscosity of the fracturing fluid composing the lamella, such as polymeric structures.

The fracturing fluids, such as the gelled fracturing fluids, disclosed herein may comprise nonaqueous gelling agents. The gelling agents include but are not limited to hydrocarbon soluble copolymers, phosphate esters, organo-metallic complex cross-linkers, amine carbamates, aluminum soaps, cocoamine (C12-C14), sebacoyl chloride, oley (C18) amine, toulen-2, 4-diisocyanate, tolune-2, 6-diisolcyanate, and combinations thereof.

The fracturing fluids, such as the hydrocarbon foam, the emulsion based foam, the emulsion, and the gelled fracturing fluids, disclosed herein may comprise secondary fluids. The secondary fluids include but are not limited to aromatics, alkanes, crude oils, and combinations thereof. The secondary fluid may comprises 10% or less by volume of the fracturing fluids described herein. The aromatics may comprise at least one of benzene, naphtha, xylene, toluene, fuel oils, olefins, and diesel. The alkanes may comprise at least one of heptane, octane, and hexane. The crude oil may comprise at least one of NGL's, condensate, light oil, and medium oil.

The fracturing fluids disclosed herein may comprise an unfractionated hydrocarbon mixture comprising ethane, propane, butane, isobutane, and pentane plus, wherein the ethane, propane, and butane comprise at least 75% by volume of the unfractionated hydrocarbon mixture.

The fracturing fluids disclosed herein may comprise an unfractionated hydrocarbon mixture comprising ethane, propane, butane, isobutane, and pentane plus, wherein the ethane comprises at least 3% by volume of the unfractionated hydrocarbon mixture.

The fracturing fluids may comprise an unfractionated hydrocarbon mixture comprising ethane, propane, butane, isobutane, and pentane plus, wherein the pentane plus comprises less than 30% by volume of the unfractionated hydrocarbon mixture.

The foamed fracturing fluids disclosed herein may be formed with any type of gas, such as carbon dioxide, nitrogen, natural gas, methane, LNG, and/or ethane, and include one or more foaming agents, such as a surfactant, to form a hydrocarbon foam or an emulsion based foam. The gas content of the foamed fracturing fluid may be between about 55% to about 95% by volume. The nitrogen content of a hydrocarbon or emulsion based foam created by any of the systems disclosed herein may be greater than 50% by volume, and the carbon dioxide content of a hydrocarbon or emulsion based foam created by any of the systems disclosed herein may be greater than 35% by volume, which causes the resulting gaseous mixtures to be outside the Flammability Limit, sometimes referred to as the Explosion Limit in which a flammable substance such as Y-Grade NGL in the presence of air can produce a fire or explosion when an ignition source such as a spark or open flame is present.

The fracturing fluids disclosed herein may be injected into a subsurface formation at a low temperature, such as at or below about 0 degrees Fahrenheit, for example as low as −30 degrees Fahrenheit.

A method of fracturing a subsurface formation, such as a hydrocarbon bearing reservoir, comprises mixing a proppant, Y-Grade NGL, and at least one of a foaming agent, an emulsifying agent, and a gelling agent and to form a fracturing fluid; increasing the pressure of the fracturing fluid using one or more high pressure pumps; and injecting the fracturing fluid into the subsurface formation at a temperature at or below about 0 degrees Fahrenheit to fracture the subsurface formation.

A method of fracturing a hydrocarbon bearing reservoir comprises mixing Y-Grade NGL, a foaming agent, optionally a foam stabilizer, a proppant, and a gas, such as nitrogen, to form a hydrocarbon foam; and pumping the hydrocarbon foam into a hydrocarbon bearing reservoir via a wellhead to fracture the hydrocarbon bearing reservoir.

A method of fracturing a hydrocarbon bearing reservoir comprises mixing Y-Grade NGL, a surfactant, water, a proppant, and a gas, such as nitrogen, to form an emulsion based foam; and pumping the emulsion based foam into a hydrocarbon bearing reservoir via a wellhead to fracture the hydrocarbon bearing reservoir.

A method of fracturing a hydrocarbon bearing reservoir comprises mixing Y-Grade NGL, an emulsifying agent, water, and a proppant to form an emulsion; and pumping the emulsion into a hydrocarbon bearing reservoir via a wellhead to fracture the hydrocarbon bearing reservoir.

A method of fracturing a hydrocarbon bearing reservoir comprises mixing Y-Grade NGL, a gelling agent, and a proppant to form a gelled fracturing fluid; and pumping the gelled fracturing fluid into a hydrocarbon bearing reservoir via a wellhead to fracture the hydrocarbon bearing reservoir.

A method of fracturing a hydrocarbon bearing formation reservoir comprises the use of Y-Grade NGL in a conventional water base fracturing system such as “slick water” or a polymer system as part of the “break down” fluid or slug to alter the wettability of the formation rock and solubilizing the organic components within the formation.

While the foregoing is directed to certain embodiments, other and further embodiments may be devised without departing from the basic scope thereof, and the scope thereof is determined by the claims that follow.

1. A method of optimizing a Y-Grade NGL fracturing fluid, comprising:
gathering geostatic data and reservoir fluid data of a hydrocarbon bearing reservoir;
assessing availability of a supply of Y-Grade NGL and a gas;
using the reservoir fluid data and data regarding the composition of the Y-Grade NGL and the gas to determine an equation of state;
generating a hydrocarbon foam through a foam generation module, wherein the foam generation module includes customizing a surfactant to be mixed with the Y-Grade NGL and the gas to form the hydrocarbon foam, adjusting foam stability of the hydrocarbon foam, customizing the hydrocarbon foam, and determining a foam rheology of the hydrocarbon foam;
formulating computational algorithms for the equation of state and the foam rheology;
formulating a 3-D frac simulation model as represented by the geostatic data and the computational algorithms to simulate a hydraulic fracturing process of the reservoir using the hydrocarbon foam; and
running multiple simulations for different hydrocarbon foams generated by the foam generation module to obtain an optimum propped frac length.

2. The method of claim 1, further comprising customizing the surfactant by selecting at least one of a siloxane surfactant, a fluorosurfactant, a fatty acid ester, a glyceride, a silicon emulsifier, and a hydrophobic silica powder as the surfactant.

3. The method of claim 1, further comprising customizing the surfactant by at least one of adjusting the molecular weight of the surfactant, selecting a surfactant that is soluble in light hydrocarbons, and adjusting the concentration of surfactant by up to 5% by weight of the liquid phase of the hydrocarbon foam.

4. The method of claim 1, further comprising adjusting foam stability by performing at least one of the following steps: altering foam quality based on the amount of the gas used to form the hydrocarbon foam, adding nanoparticles to reduce fluid loss of the liquid phase of the hydrocarbon foam, adding a hydrocarbon soluble co-polymer to viscosify the liquid phase of the hydrocarbon foam, and changing the type of gas used to form the hydrocarbon foam.

5. The method of claim 1, further comprising adjusting foam stability by adjusting apparent viscosity, wherein the apparent viscosity is adjusted by performing at least one of the following steps: altering foam quality based on the amount of the gas used to form the hydrocarbon foam, adding a hydrocarbon soluble co-polymer to viscosify the liquid phase of the hydrocarbon foam, and adding a secondary fluid comprising up to 10% of the liquid phase of the hydrocarbon foam, wherein the secondary fluid comprises at least one of aromatics, alkanes, and crude oil.

6. The method of claim 1, further comprising customizing the hydrocarbon foam by adding a secondary fluid to the hydrocarbon foam, wherein the secondary fluid comprises at least one of aromatics, alkanes, and crude oil.

7. The method of claim 6, wherein the crude oil comprises at least one of NGL's, condensate, light oil, and medium oil.

8. The method of claim 1, further comprising determining the foam rheology based on apparent viscosity, density, friction, flow rate, fluid loss, shear, and heat loss as a function of temperature, pressure, and composition of the hydrocarbon foam.

9. The method of claim 1, wherein the gas comprises at least one of nitrogen, carbon dioxide, natural gas, methane, LNG, and ethane.

10. The method of claim 1, wherein the Y-Grade NGL comprise an unfractionated hydrocarbon mixture comprising ethane, propane, butane, isobutane, and pentane plus, wherein the unfractionated hydrocarbon mixture is a byproduct of a condensed and de-methanized hydrocarbon stream, wherein the unfractionated hydrocarbon mixture is condensed out of the hydrocarbon stream at a temperature at or below 0 degrees Fahrenheit, wherein the unfractionated hydrocarbon mixture comprises ethane, propane, and butane in an amount of at least 75% by volume, and wherein the unfractionated hydrocarbon mixture comprises pentane plus in an amount less than 30% by volume.

11. The method of claim 1, wherein running multiple simulations comprises generating different hydrocarbon foams though the foam generation module, formulating computational algorithms for each hydrocarbon foam based on the equation of state and foam rheology of each hydrocarbon foam, and formulating 3-D frac simulation models as represented by the geostatic data and the computational algorithms to simulate a hydraulic fracturing process of the reservoir using each hydrocarbon foam to gather enough data to determine the optimum propped frac length and which hydrocarbon foam will achieve the optimum propped frac length.

12. A method of optimizing a Y-Grade NGL fracturing fluid, comprising:
gathering geostatic data and reservoir fluid data of a hydrocarbon bearing reservoir;
assessing availability of a supply of Y-Grade NGL, a gas, and water;
using the reservoir fluid data and data regarding the composition of the Y-Grade NGL, the gas, and the water to determine an equation of state;
generating an emulsion based foam through an emulsion based foam generation module, wherein the emulsion based foam generation module includes customizing a surfactant to be mixed with the Y-Grade NGL, the gas, and the water to form the emulsion based foam, adjusting foam stability of the emulsion based foam, customizing the emulsion based foam, and determining an emulsion based foam rheology of the emulsion based foam;
formulating computational algorithms for the equation of state and the emulsion based foam rheology;
formulating a 3-D frac simulation model as represented by the geostatic data and the computational algorithms to simulate a hydraulic fracturing process of the reservoir using the emulsion based foam; and
running multiple simulations for different emulsion based foams generated by the emulsion based foam generation module to obtain an optimum propped frac length.

13. The method of claim 12, wherein the surfactant that acts as a foaming agent, an emulsifying agent, or both.

14. The method of claim 12, further comprising customizing the surfactant by at least one of adjusting the molecular weight of the surfactant and adjusting the concentration of surfactant by up to 5% by weight of the liquid phase of the emulsion based foam.

15. The method of claim 12, further comprising customizing the surfactant by selecting at least one of a non-ionic surfactant, an anionic surfactant, and a cationic surfactant as the surfactant, wherein the non-ionic surfactant is soluble in light hydrocarbons, and wherein the anionic surfactant and the cationic surfactants are soluble in water.

16. The method of claim 15, wherein the non-ionic surfactant comprises at least one of a siloxane, a fluorosurfactant, a fatty acid ester, a glyceride, a silicon emulsifier, and a hydrophobic silica powder.

17. The method of claim 12, further comprising adjusting foam stability by performing at least one of the following steps: altering foam quality based on the amount of the gas used to form the emulsion based foam, adding nanoparticles to reduce fluid loss of the liquid phase of the emulsion based foam, adding a hydrocarbon soluble co-polymer to viscosify the liquid phase of the emulsion based foam, adding a water soluble co-polymer to viscosify the liquid phase of the emulsion based foam, and changing the type of gas used to form the emulsion based foam.

18. The method of claim 12, further comprising further comprising adjusting foam stability by adjusting apparent viscosity, wherein the apparent viscosity is adjusted by performing at least one of the following steps: altering foam quality based on the amount of the gas used to form the emulsion based foam, adding a hydrocarbon soluble co-polymer to viscosify the liquid phase of the emulsion based foam, adding a water soluble co-polymer to viscosify the liquid phase of the emulsion based foam, and adding a secondary fluid comprising up to 10% of the liquid phase of the emulsion based foam, wherein the secondary fluid comprises at least one of aromatics, alkanes, and crude oil.

19. The method of claim 12, further comprising customizing the emulsion based foam by adding a secondary fluid to the emulsion based foam, wherein the secondary fluid comprises at least one of aromatics, alkanes, and crude oil.

20. The method of claim 19, wherein the crude oil comprises at least one of NGL's, condensate, light oil, and medium oil.

21. The method of claim 12, further comprising determining the foam rheology based on apparent viscosity, density, friction, flow rate, fluid loss, shear, and heat loss as a function of temperature, pressure, and composition of the emulsion based foam.

22. The method of claim 12, wherein the water is at least one of brine, seawater, and formation water and comprises up to 10% of the liquid phase of the emulsion based foam.

23. The method of claim 12, wherein the water is fresh water inhibited with potassium chloride and comprises up to 10% of the liquid phase of the emulsion based foam, wherein the fresh water inhibited with potassium chloride comprises up to 4% potassium chloride.

24. The method of claim 12, wherein the gas comprises at least one of nitrogen, carbon dioxide, natural gas, methane, LNG, and ethane.

25. The method of claim 12, wherein the Y-Grade NGL comprise an unfractionated hydrocarbon mixture comprising ethane, propane, butane, isobutane, and pentane plus, wherein the unfractionated hydrocarbon mixture is a byproduct of a condensed and de-methanized hydrocarbon stream, wherein the unfractionated hydrocarbon mixture is condensed out of the hydrocarbon stream at a temperature at or below 0 degrees Fahrenheit, wherein the unfractionated hydrocarbon mixture comprises ethane, propane, and butane in an amount of at least 75% by volume, and wherein the unfractionated hydrocarbon mixture comprises pentane plus in an amount less than 30% by volume.

26. The method of claim 12, wherein running multiple simulations comprises generating different emulsion based foams through the emulsion based foam generation module, formulating computational algorithms for each emulsion based foam based on the equation of state and foam rheology of each emulsion based foam, and formulating 3-D frac simulation models as represented by the geostatic data and the computational algorithms to simulate a hydraulic fracturing process of the reservoir using each emulsion based foam to gather enough data to determine the optimum propped frac length and which emulsion based foam will achieve the optimum propped frac length.

27. A method of optimizing a Y-Grade NGL fracturing fluid, comprising:
gathering geostatic data and reservoir fluid data of a hydrocarbon bearing reservoir;
assessing availability of a supply of Y-Grade NGL and water;
using the reservoir fluid data and data regarding the composition of the Y-Grade NGL and the water to determine an equation of state;
generating an emulsion through an emulsion generation module, wherein the emulsion generation module includes customizing a surfactant to be mixed with the Y-Grade NGL and the water to form the emulsion, adjusting emulsion stability of the emulsion, customizing the emulsion, and determining an emulsion rheology of the emulsion;
formulating computational algorithms for the equation of state and the emulsion rheology;
formulating a 3-D frac simulation model as represented by the geostatic data and the computational algorithms to simulate a hydraulic fracturing process of the reservoir using the emulsion; and
running multiple simulations for different emulsions generated by the emulsion generation module to obtain an optimum propped frac length.

28. The method of claim 27, further comprising customizing the surfactant by at least one of adjusting the molecular weight of the surfactant and adjusting the concentration of surfactant by up to 5% by weight of the emulsion.

29. The method of claim 27, further comprising customizing the surfactant by selecting at least one of a non-ionic surfactant, an anionic surfactant, and a cationic surfactant as the surfactant, wherein the non-ionic surfactant is soluble in light hydrocarbons, and wherein the anionic surfactant and the cationic surfactants are soluble in water.

30. The method of claim 29, wherein the non-ionic surfactant comprises at least one of a siloxane surfactant, a fluorosurfactant, a fatty acid ester, a glyceride, a silicon emulsifier, and a hydrophobic silica powder as the surfactant.

31. The method of claim 27, further comprising adjusting emulsion stability by performing at least one of the following steps: changing the percent volume of water used to form the emulsion, and adding a viscosifier to the emulsion.

32. The method of claim 31, wherein the viscosifier comprises at least one of a hydrocarbon soluble co-polymer and a water soluble viscosifier, and wherein the water soluble viscosifer comprises at least one of water soluble co-polymers, polysaccarides, guar gum, viscoelastic surfactants, crosslinkers, cellulosic viscosifiers, and hydroxyethyl cellulos.

33. The method of claim 27, further comprising adjusting emulsion stability by adjusting the apparent viscosity, wherein the apparent viscosity is adjusted by performing at least one of the following steps: adding a hydrocarbon soluble co-polymer to viscosify the liquid phase of the emulsion, adding a water soluble co-polymer to viscosify the liquid phase of the emulsion, changing the percent volume of water used to form the emulsion, and adding a secondary fluid comprising up to 10% of the liquid phase of the emulsion, wherein the secondary fluid comprises at least one of aromatics, alkanes, and crude oil.

34. The method of claim 27, further comprising customizing the emulsion by adding a secondary fluid to the emulsion, wherein the secondary fluid comprises at least one of aromatics, alkanes, and crude oil.

35. The method of claim 34, wherein the crude oil comprises at least one of NGL's, condensate, light oil, and medium oil.

36. The method of claim 27, further comprising determining the emulsion rheology based on apparent viscosity, density, friction, flow rate, fluid loss, shear, and heat loss as a function of temperature, pressure, and composition of the emulsion.

37. The method of claim 27, wherein the water is at least one of brine, seawater, and formation water, and comprises up to 10% of the liquid phase of the emulsion.

38. The method of claim 27, wherein the water is fresh water inhibited with potassium chloride and comprises up to 10% of the liquid phase of the emulsion, wherein the fresh water inhibited with potassium chloride comprises up to 4% potassium chloride.

39. The method of claim 27, wherein the Y-Grade NGL comprise an unfractionated hydrocarbon mixture comprising ethane, propane, butane, isobutane, and pentane plus, wherein the unfractionated hydrocarbon mixture is a byproduct of a condensed and de-methanized hydrocarbon stream, wherein the unfractionated hydrocarbon mixture is condensed out of the hydrocarbon stream at a temperature at or below 0 degrees Fahrenheit, wherein the unfractionated hydrocarbon mixture comprises ethane, propane, and butane in an amount of at least 75% by volume, and wherein the unfractionated hydrocarbon mixture comprises pentane plus in an amount less than 30% by volume.

40. The method of claim 27, wherein running multiple simulations comprises generating different emulsions though the emulsion generation module, formulating computational algorithms for each emulsion based on the equation of state and emulsion rheology of each emulsion, and formulating 3-D frac simulation models as represented by the geostatic data and the computational algorithms to simulate a hydraulic fracturing process of the reservoir using each emulsion to gather enough data to determine the optimum propped frac length and which emulsion will achieve the optimum propped frac length.

41. A method of optimizing a Y-Grade NGL fracturing fluid, comprising:
gathering geostatic data and reservoir fluid data of a hydrocarbon bearing reservoir;
assessing availability of a supply of Y-Grade NGL;
using the reservoir fluid data and data regarding the composition of the Y-Grade NGL to determine an equation of state;
generating a gelled fracturing fluid through a gel generation module, wherein the gel generation module includes customizing a gelling agent to be mixed with the Y-Grade NGL to form the gelled fracturing fluid, adjusting gel stability of the gelled fracturing fluid, customizing the gelled fracturing fluid, and determining a gel rheology of the gelled fracturing fluid;
formulating computational algorithms for the equation of state and the gel rheology;
formulating a 3-D frac simulation model as represented by the geostatic data and the computational algorithms to simulate a hydraulic fracturing process of the reservoir using the gelled fracturing fluid; and
running multiple simulations for different gelled fracturing fluids generated by the gel generation module to obtain an optimum propped frac length.

42. The method of claim 41, further comprising customizing the gelling agent by selecting at least one of hydrocarbon soluble copolymers, phosphate esters, organo-metallic complex cross-linkers, amine carbamates, aluminum soaps, cocoamine (C12-C14), sebacoyl chloride, oley (C18) amine, toulen-2, 4-diisocyanate, tolune-2, 6-diisolcyanate, and combinations thereof as the gelling agent.

43. The method of claim 41, further comprising adjusting gel stability by changing at least one of the type of gelling agent and the concentration of the gelling agent.

44. The method of claim 41, further comprising customizing the gelled fracturing fluid by adding a secondary fluid to the gelled fracturing fluid, wherein the secondary fluid comprises at least one of aromatics, alkanes, and crude oil.

45. The method of claim 44, wherein the crude oil comprises at least one of NGL's, condensate, light oil, and medium oil.

46. The method of claim 41, further comprising determining the gel rheology based on apparent viscosity, density, friction, flow rate, fluid loss, shear, and heat loss as a function of temperature, pressure, and composition of the gelled fracturing fluid.

47. The method of claim 41, wherein the Y-Grade NGL comprise an unfractionated hydrocarbon mixture comprising ethane, propane, butane, isobutane, and pentane plus, wherein the unfractionated hydrocarbon mixture is a byproduct of a condensed and de-methanized hydrocarbon stream, wherein the unfractionated hydrocarbon mixture is condensed out of the hydrocarbon stream at a temperature at or below 0 degrees Fahrenheit, wherein the unfractionated hydrocarbon mixture comprises ethane, propane, and butane in an amount of at least 75% by volume, and wherein the unfractionated hydrocarbon mixture comprises pentane plus in an amount less than 30% by volume.

48. The method of claim 41, wherein running multiple simulations comprises generating different gelled fracturing fluids though the gel generation module, formulating computational algorithms for each gelled fracturing fluid based on the equation of state and gel rheology of each gelled fracturing fluid, and formulating 3-D frac simulation models as represented by the geostatic data and the computational algorithms to simulate a hydraulic fracturing process of the reservoir using each gelled fracturing fluid to gather enough data to determine the optimum propped frac length and which gelled fracturing fluid will achieve the optimum propped frac length.
